(19) United States
(12) Patent Application Publication
(10) Pub. No.: US 2025/0127738 A1
(43) Pub. Date: Apr. 24, 2025

(54) DECALCIFICATION COMPOSITION FOR TREATMENT AND/OR PREVENTION OF CARDIOVASCULAR DISEASE

(71) Applicant: Murray Henderson Williams, Windcrest, TX (US)

(72) Inventor: Murray Henderson Williams, Windcrest, TX (US)

(21) Appl. No.: 18/491,163

(22) Filed: Oct. 20, 2023

(51) Int. Cl. A61K 31/194; A61K 31/198; A61K 31/375

(52) U.S. Cl. CPC A61K 31/194; A61K 31/198; A61K 31/375

(57) ABSTRACT

Disclosed are decalcification compositions for the treatment and prevention of cardiovascular disease and methods of administering such compositions to patients.

### FIELD

The present subject matter relates in general to a decalcifying composition for treatment and/or prevention of cardiovascular disease and, more particularly, to decalcifying compositions including one or more naturally derived chelating agents.

### BACKGROUND

Atherosclerosis is a pattern of the disease arteriosclerosis in which an arterial wall develops abnormalities, called lesions. (Arteriosclerosis/Atherosclerosis—Symptoms and Causes, Mayo Clinic.) The lesions may lead to narrowing due to a buildup of atheromatous plaque. (What is Atherosclerosis?—NHLBI, NIH, 2016; “Cyclic phosphatidic acid inhibits secretion of vascular endothelial growth factor from diabetic human coronary endothelial cells through peroxisome proliferator-activated receptor gamma,” Molecular and Cellular Endocrinology, 412:320-329, 2015.) At onset there are usually no symptoms, but if they develop, symptoms generally begin around middle age. When severe, it can result in coronary artery disease, stroke, peripheral artery disease, or kidney problems, depending on the arteries affected.

Arteriosclerosis is the thickening, hardening, and loss of elasticity of the walls of arteries. (Dorland Medical Dictionary.) This process gradually restricts the blood flow to one's organs and tissues and can lead to severe health risks brought on by atherosclerosis, which is a specific form of arteriosclerosis caused by the buildup of fatty plaques, cholesterol, and other substances in and on the artery walls.

US 2006/0083727 to Kajander et al. discloses compositions said to include a nutraceutical supplement, an antibiotic, and a metal chelating agent administered to patients for treating and/or venting pathological calcification and/or plaque formation. Also disclosed are methods involving administering a therapeutically effective nutraceutical supplement, tetracycline HCL, and EDTA (ethylenediamine tetra-acetic acid) calcium di-sodium salt to patients for preventing and/or treating calcific disease.

U.S. Pat. No. 6,423,742 to Larson discloses compositions for reducing vascular plaque formation. US 2016/0220520 to Beaudin et al. is directed to biocompatible solvents, systems, and methods for treatment of atherosclerosis and heart disease.

Cardiovascular diseases—whether arteriosclerosis or atherosclerosis—are known to result in a hardening of arteries, the number one killer of people in the western world. Treatment includes expensive and/or ineffective drugs and surgery.

While drugs that reduce cholesterol and blood pressure may only treat symptoms, not underlying causes, and may come with a host of harmful side effects, EDTA (ethylenediamine tetra-acetic acid) infusions, often used to decalcify vascular systems, are expensive and known to be inconvenient in terms of comfort and time.

Additional problem-solving attempts include general surgery (which is an invasive and short-term “fix” for the root-problem cause) and bypass or stent surgery, an expensive procedure with a poor cost/benefit ratio (that only “fixes” a small area).

As can be appreciated, certain people want an inexpensive, noninvasive treatment for cardiovascular disease—without harmful side effects—to be available.

### SUMMARY

The present subject matter is directed to a composition that de-calcifies a calcified vascular system and provides a treatment method for de-calcifying a calcified vascular system of people who have calcified vascular systems, resulting in the reversal of their calcified vascular systems, while also repairing their arteries.

The present subject matter, an effective and inexpensive alternative to drugs and surgery for cardiovascular disease, is a composition comprising sodium citrate (with or without, vitamin C and L-lysine or other ingredients). The composition provides an effective and inexpensive alternative for treating cardiovascular disease.

The present subject matter makes use of “chelation therapy” to de-calcify blood vessels by using non-toxic organic ingredients. The present treatment method addresses the underlying cause of cardiovascular disease in a non-invasive way that works better than drugs, surgery, and other chelation substances, including EDTA.

The composition of the present subject matter may also be used in cases of organ failure and possibly cancer to de-calcify blood vessels feeding those organs.

### BRIEF DESCRIPTION OF THE DRAWING FIGURES

FIG. 1 presents a chemical equation, in organic chemical format, showing benefits of compositions manufactured using principles of the present subject matter.

FIG. 2 depicts a method of formulating and using the present subject matter.

### DETAILED DESCRIPTION

The following detailed description consists essentially of only currently contemplated modes of carrying out exemplary embodiments of the present subject matter. The description is thus not to be taken in a limiting sense but rather is made merely for purposes of illustrating general principles of the present subject matter, since the scope of the present subject matter is best defined by the appended claims.

In a broad sense, one aspect of the present subject matter includes methods by which patients take predetermined amounts of compositions consisting of select ingredients. Compositions of the present subject matter include a naturally derived chelating agent, a naturally derived vasodilator, and a naturally derived healing ingredient. Methods of treating or preventing cardiovascular disease include administering to a patient a predetermined effective amount of such a composition.

Sodium citrate, a chelating active ingredient, provides sodium-buffered citric acid which, by itself, is unpleasant to one's stomach. The sodium buffer enables the composition to be gentle on a subject patient's stomach upon oral administration.

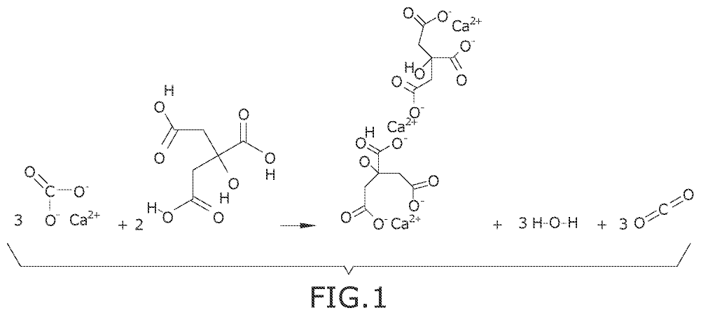

Citric acid naturally reacts with calcium carbonate triggering a chemical reaction that produces calcium citrate, carbon dioxide and water, as represented in FIG. 1 and as depicted in the chemical equation presented immediately below:

$$3 CaCO 3 + 2 H 3 C 6H5O7 \to Ca 3 ( C 6H5O7 ) 2 + 3 H 2 O + 3 CO 2$$

The citrate ion, believed to bind to calcium ions in artery plaque, removes limescale and softens plaque. Without limescale, artery plaque may be naturally dissolved by the bloodstream. A possible reason why remaining plaques dissolve after limescale removal may be due to the alkalinity of the blood, which is normally 7.4 on the pH scale. Alkaline substances like soap and sodium hydroxide break down fatty substances, which are slightly acidic.

Ascorbic acid (vitamin C), a secondary chelator, though not as potent as citric acid, is also believed to work as a vasodilator, to dilate the blood vessels and allow for easier blood flow. Lysine is believed to increase calcium absorption in the gut and to reduce calcium loss in urine, as well as to prevent the building of calcium in blood vessels. Ascorbic acid and lysine are believed to work together to repair damaged blood vessel walls, especially in the heart, and are a healing component.

In embodiments of the invention, certain other organic ingredients (e.g., vitamins, minerals and amino acids) may be added in lieu of ascorbic acid and lysine.

Ingredients of the present subject matter may be derived from natural sources. In some embodiments ingredients may be derived from organic sources.

In embodiments, compositions of the present subject matter may be provided in capsule form for oral administration. In other embodiments, the inventive composition may be provided in a pill form with binders holding ingredients together.

The present subject matter includes methods of decalcifying a subject patient's arteries. Such methods comprise administering a composition containing predetermined amounts of sodium citrate effective to decalcify the patient's arteries.

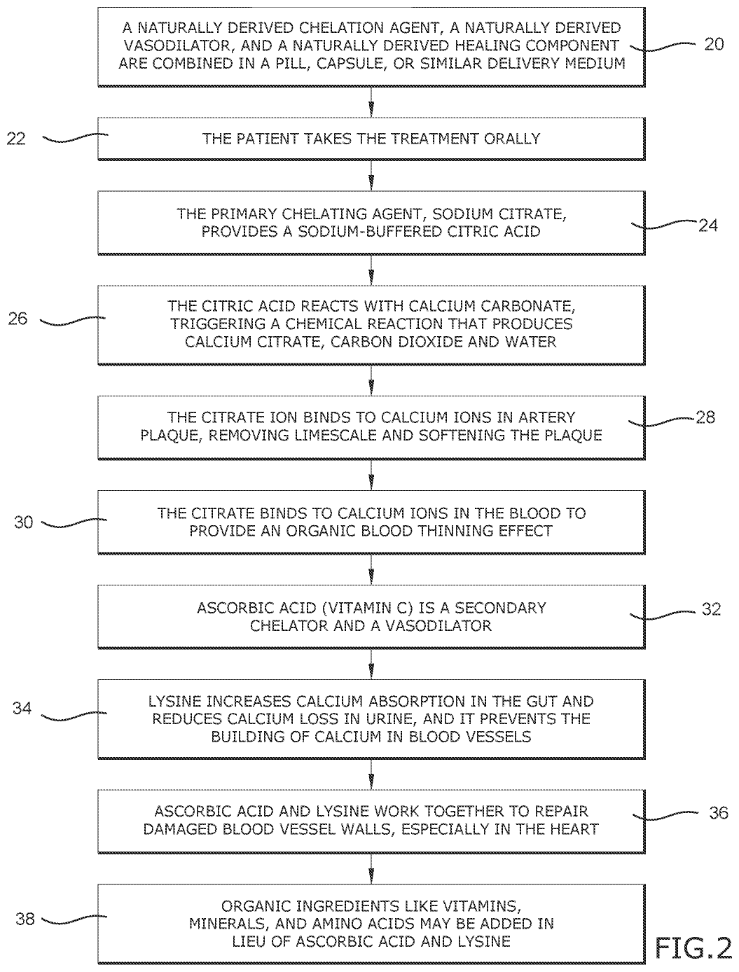

Please refer to FIG. 2 for a detailed description of additional aspects and features of the present subject matter. At box 20: A chelating agent, a vasodilator, and a healing component, each naturally derived and present in a predetermined amount, are combined, and thereafter incorporated into a pill, a capsule, or another ingredient-delivery medium administered to a subject patient orally or intravenously. At box 22: An embodiment of the present subject matter is administered, e.g., orally to a subject patient as a treatment for and/or preventative of cardiovascular disease. At box 24: A primary and preferred chelating agent, sodium citrate, is said to provide a sodium-buffered citric acid ingredient for a composition of the present subject matter. At box 26, the citric acid is said to react (see FIG. 1) with calcium carbonate to trigger a chemical reaction, producing calcium citrate, carbon dioxide, and water. At box 28: The citrate ion is said to bind to calcium ions in artery plaque, thereby removing limescale and softening the plaque. At box 30: The citrate thus produced is said to bind to calcium ions in the blood to provide an organic blood-thinning effect. At box 32: The ascorbic acid ingredient (e.g., vitamin C) is said to be a secondary chelator, and a vasodilator. At box 34: The lysine ingredient is said to increase calcium absorption in the gut (of a subject patient), for reducing calcium loss in urine (of subject patients) and preventing accumulation of calcium in patient blood vessels. At box 36: The ascorbic acid and lysine are said to work together to repair damaged blood vessel walls, especially in the heart of a subject patient. At box 38: It is stated that organic ingredients (e.g., vitamins, minerals, and amino acids) may be added in lieu of ascorbic acid and lysine to select compositions of the present subject matter.

This application discloses a decalcification composition and method. The present subject matter is described with reference to exemplary embodiments. Thus, alternatives, changes, or modifications will become apparent to a person of ordinary skill in the art (“POSITA”) after this description and the figs are reviewed. Therefore, alternatives, changes, and modifications are to be treated as forming a part of the present subject matter insofar as they fall within the scope of the appended claims.

What is claimed is:

1. A decalcification composition for treatment and/or prevention of cardiovascular disease in a patient, comprising sodium citrate for producing citrate ions in an amount effective for binding to calcium ions in artery plaque of a patient.

2. The decalcification composition of claim 1, also including ascorbic acid and lysine.

3. The decalcification composition of claim 1, also including vitamins and minerals.

4. The decalcification composition of claim 3, further including amino acids.

5. The decalcification composition of claim 1, incorporated into a pill.

6. The decalcification composition of claim 1, incorporated into a capsule.

7. A method for treating and/or preventing cardiovascular disease in a patient comprising administering to the patient a decalcification composition in an amount effective for treating and/or preventing the cardiovascular disease, wherein the composition comprises sodium citrate for producing citrate ions in an amount effective to bind to calcium ions in artery plaque of the patient to remove limescale in an artery of the patient and soften plaque therein, due to blood flow of the patient.

8. The method of claim 7, wherein the composition includes ascorbic acid and lysine.

9. The method of claim 7, wherein the composition includes vitamins and minerals.

10. The method of claim 9, wherein the composition further includes amino acids.

11. The method of claim 7, wherein the composition is administered orally.

12. The method of claim 7, wherein the composition is administered intravenously.
